(19) United States
(12) Patent Application Publication
(10) Pub. No.: US 2008/0065063 A1
(43) Pub. Date: Mar. 13, 2008

(54) HIGH-FREQUENCY ENDOSCOPIC INSTRUMENT

(75) Inventor: Thomas Wosnitza, Luneburg (DE)

Correspondence Address: RANKIN, HILL & CLARK LLP, 38210 Glenn Avenue, WILLOUGHBY, OH, 44094-7808, US

(73) Assignee: OLYMPUS WINTER & IBE GMBH, Hamburg (DE)

(21) Appl. No.: 11/848,969

(22) Filed: Aug. 31, 2007

(30) Foreign Application Priority Data
Sep. 7, 2006 (DE) 10 2006 041 919.7

(51) Int. Cl. A61B 18/14

(52) U.S. Cl. 606/46

(57) ABSTRACT

A high-frequency (hf) endoscopic instrument (1) through which runs a metal stem tube (3) enclosing an operative conduit (10), fitted with a longitudinally displaceable electrode (13) supported in the operative conduit (10) and connected by an electric lead (14) crossing said operative conduit (10) to one of the two terminals of an hf generator (18). The metal stem tube (3) is connected to the other terminal of the generator (18) and in that the distal end zone of the operative conduit (10) is enveloped by an insulating tube (19) projecting beyond the distal end of the operative conduit (10) by a distance which corresponds to the safety separation (20) between the terminals in an electrically conductive liquid.

### BACKGROUND OF THE INVENTION

The present invention relates to a high-frequency (hf) endoscopic instrument.

Such hf instruments are used in treating body tissue using an electrode current. Illustratively, tumors may be removed in this manner from the bladder wall. The surgery site may be reached by displacing the electrode in the operative conduit. The metal stem as a simple tube, per se, may constitute the operative conduit. However the operative conduit also may be configured within an instrument enclosed by the metal stem, for instance a cystoscope, wherein the metal stem not only receives the operative conduit, but also an optics and a light guide such as an optic fiber in order to observe the illuminated surgery site during surgery.

Known hf instruments of this kind are fitted with a neutral electrode connected to the second hf generator terminal and externally applied to the patient's body, so that the hf current may pass from the active electrode through the body tissue to the neutral electrode. The liquid-filled space receiving the electrode, for instance the human bladder, is filled in such a case with a liquid of low electrical conductance.

HF instruments outside the above species disclosed in WO 97/00647 use a bipolar electrode system which is fitted with the two electrodes apart from each other, mutually insulated and respectively connected to one of the generator's two output terminals. Such a design is used for highly electrically conductive saline-enriched liquids, whereby the current passes directly between the electrodes and through the liquid. This procedure eliminates the injurious current through the body. However this latter design is more elaborate and costlier.

### BRIEF SUMMARY OF THE INVENTION

The object of the present invention is to create an hf instrument of simple design which allows bipolar treatment in conductive liquids.

In the present invention, a commercially available monopolar needle electrode may be used which is, for instance, a conventional conductive wire insulated over most of its length while being bare at its tip at the electrode proper. This electrode is used in a plain metal tube or illustratively in a cystoscope fated with an operative conduit, the metal stem being electrically connected to the second hf generator terminal. Accordingly, the current is set up between the electrode and the metal stem tube across a short path through the conductive liquid. However such a design raises a problem of electrical safety because when displaced longitudinally, the electrode may reach the vicinity of the distal mouth of the operative conduit, where it then might be a few tenths of a mm from the instrument's metal stem tube which is connected to the second terminal. In an electrically highly conducting liquid, a strong electric arc might then be generated entailing an electric short and possibly welding the stem to the electrode. The present invention offers palliation by means of the insulating tube which is configured at the distal end of the operative conduit and which distally projects beyond it a distance such that when retracting the electrode, the minimum separation between electrode and stem precluding an electric short shall be maintained.

It is sufficient that the insulating tube be configured only near the distal end of the treatment tube to preclude a short when current is applied in the treatment range of the electrode wherein it is advanced and retracted. However, advantageously, the insulating tube shall run the full length of the operative conduit, whereby assembly is made simpler and moreover the electrode need not be insulated over its full length.

Illustratively, the insulating tube may be a ceramic tube, though advantageously, it may be designed more simply and more economically as an appropriately insulating plastic sheath.

The present invention is shown in illustrative and schematic manner in the appended drawings.

### BRIEF DESCRIPTION OF THE DRAWINGS

FIG. 1 is a sideview of a cystoscope of the present invention,

FIG. 2 is a cross-section along line 2-2 of FIG. 1, and

FIG. 3 is a section along line 3-3 of FIG. 2.

### DETAILED DESCRIPTION OF THE INVENTION

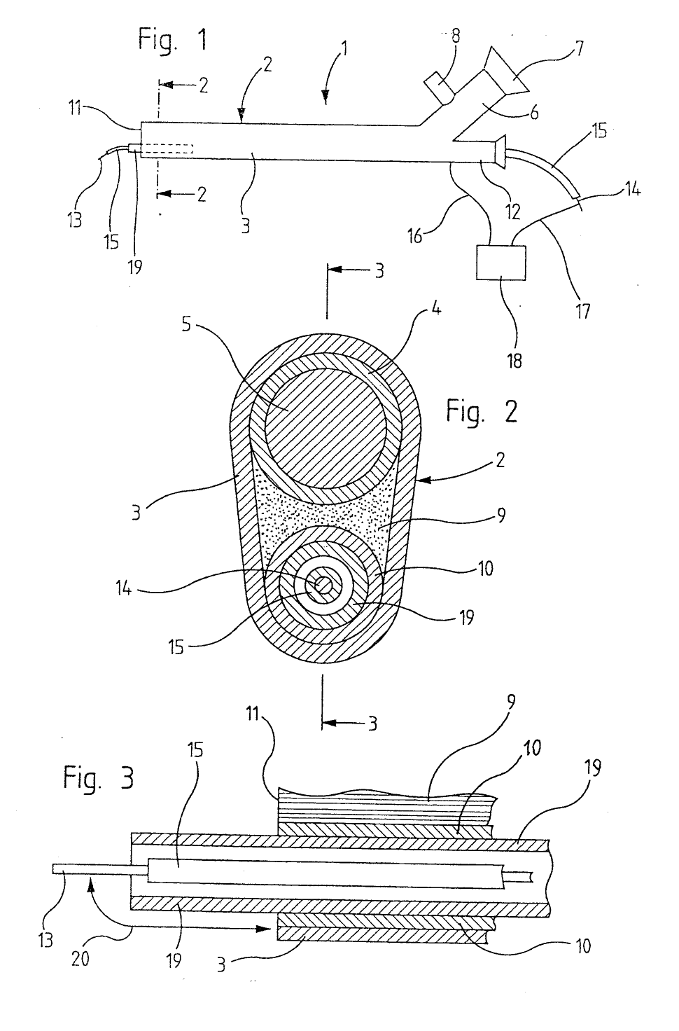

The hf endoscopic instrument 1 shown in FIGS. 1-3 comprises a stem 2 shown in cross-section in FIG. 2. The outer part of the stem 2 is a metal stem tube 3. An optics 5 is configured in an optics tube 4 within the stem. This optics may be a fiber optics or a lens optics which at its distal end is fitted with an objective and leading to an ocular 7 within an oblique proximal end segment 6. A video optics also may be used. The end segment 6 moreover is fitted with a light guide hookup stub 8 from which a fiber optics 9 runs through the free cross-sectional parts in the metal stem tube 3 as far as the end face of this metal stem tube 3.

An operative conduit in the form of a tube 10 is also configured within the metal stem tube 3 and issues at the distal end into the end face 11 of the stem 2 while issuing at the proximal end into an input stub 12.

A needle electrode 13 runs through the operative conduit constituted in the duct 10 and is in the form of the free end of a conductor 14 which is fitted with an insulation 15 as far as the bared tip 13. The metal stem tube 3 and the conductor 14 of the needle electrode 13 are connected by leads 16 and 17 to the two terminals of an hf generator 18.

The instrument 1 shown in the figures, illustratively, is inserted into the human bladder in order to remove—by means of the hf-loaded needle electrode 13—tissue from the bladder wall, for instance a small tumor. Preferably, the space around the needle electrode 13 is supplied with an electrically well-conducting saline liquid. The electric current is set up between the needle electrode 13 and the metal stem tube 3. Preferably, a plasma is generated around the needle electrode 13 to remove tissue.

The needle electrode 13 is axially displaceable within the operative conduit 10 and may be axially displaced while the current is applied in order to carry out necessary surgical motions. If in the process the needle electrode 13 moves near the end face 11 of the metal stem tube 3 or near the operative conduit 10 which is electrically connected to the metal stem tube and also is metallic, said current may become large and overload the hf generator 18. Even welding may take place.

To avert costly safety precautions, an insulating tube 19 is used, which, as best seen in FIG. 3, is configured within the operative conduit 10 and projects some distance beyond the end face 11 of said operative conduit 10. If, as shown in FIG. 3, the electrified needle electrode 13 is retracted excessively, there will be danger of coming too close to the other electric terminal at the metal stem tube 3, and then it shall be screened from the segment of the insulating tube 19 projecting beyond the end face 11, as a result of which an electrically significant minimum distance shall be maintained between the needle electrode 13 and the end face 11, electrical overload being still precluded at said minimum distance. More laborious electrical safety measures are eliminated by the above simple design.

As shown in dashed lines in FIG. 1, the insulating tube 19 may be configured as a short tube stub in the distal end zone of the metal stem tube 3, though it may also be configured over the full length of the operative conduit 10. This feature makes the installation of the insulator tube 19 easier and offers a way to use the needle electrode 13 without the insulation 15, unless otherwise needed, for instance to constrain the current to the free length of the needle electrode 13.

The insulating tube 19 may be made of an appropriate insulating material such as a ceramic or it may be a plastic sheath. It may be affixed appropriately, for instance by bonding, in the operative conduit 10.

In a much simplified embodiment variation (not shown), the hf instrument may be constituted merely by the operative conduit 10 which is connected by the lead 16 to the hf generator 18. The electrode configuration 13, 14, 15 and the insulating tube 19 are mounted in the above described manner in the operative conduit 10. However such an instrument does not offer visual observation.

1. A high-frequency (hf) endoscopic instrument (1) comprising:
a metal stem tube (3) enclosing an operative conduit (10), fitted with a electrode (13) supported longitudinally displaceable in the operative conduit (10) and connected by an electric lead (14) running through said operative conduit (10) to one of the two terminals of an hf generator (18),
wherein the metal stem tube (3) is connected to the other terminal of the generator (18) and in that the distal end zone of the operative conduit (10) is lined with an insulating tube (19) projecting beyond the distal end of the operative conduit (10) by a distance which corresponds to the safety separation (20) between said terminals in an electrically conductive liquid.

2. Instrument as claimed in claim 1, wherein the insulating tube (19) runs over the full length of the operative conduit (10).

3. Instrument as claimed in claim 1, wherein the insulating tube (19) is a plastic sheath.
